(19) United States
(12) Patent Application Publication
(10) Pub. No.: US 2022/0160665 A1
(43) Pub. Date: May 26, 2022

(54) COMPOSITION IN MICRO-ENCAPSULATED FORM, BASED ON N-BUTYRICACID OR DERIVATIVES THEREOF, FOR TREATING CROHNS DISEASE OR ULCERATIVE RECTAL COLITIS

(71) Applicant: SILA S.R.L., Noale (VE) (IT)

(72) Inventors: Maurizio LORENZON, Noale (VE) (IT); Sonia FACCHIN, Torreglia (PD) (IT); Nicola VITULO, Vigonza (PD) (IT); Barbara PERINI, Padova (IT); Andrea BUDA, Feltre (BL) (IT); Fabiana ZINGONE, Padova (IT); Chiara ROMUALDI, Vigonza (PD) (IT); Renata D'INCÀ, Padova (IT); Edoardo SAVARINO, Padova (IT)

(73) Assignee: SILA S.R.L., Noale (VE) (IT)

(21) Appl. No.: 17/434,865

(22) PCT Filed: Feb. 27, 2020
(86) PCT No.: PCT/IB2020/051684

§ 371 Date: Aug. 30, 2021

(30) Foreign Application Priority Data
Mar. 1, 2019 (IT) 102019000003043

(51) Int. Cl. A61K 31/19; A61K 9/50; A61K 47/14; A61K 47/02; A61P 1/12

(52) U.S. Cl. CPC A61K 31/19; A61K 9/5015; A61P 1/12; A61K 47/02; A61K 47/14

(57) ABSTRACT

A composition in micro-encapsulated form, comprising a core based on n- butyric acid or a derivative thereof, and a coating based on a lipid matrix which surrounds the core, in which the matrix has a content of C14-C22 long chain saturated fatty acids between 40% and 95%, is used for the treatment of ulcerative rectal colitis or Crohn's disease.

### TECHNICAL FIELD

The present invention relates to a novel therapeutic use of a composition based on n-butyric acid or the derivatives thereof in micro-encapsulated form.

### TECHNOLOGICAL BACKGROUND

Crohn's disease and ulcerative rectal colitis are listed among chronic Inflammatory Bowel Diseases (IBD). These pathologies, both of an uncertain etiology, can affect in a particular manner the colon tract and the rectal intestine, causing a vast range of serious symptoms, including abdominal pains, diarrhoea (including haematic), vomiting, loss of weight. Often, there also occur important complications in other extra-intestinal organs, such as, for example, peripheral arthritis, ankylosing spondylitis, uveitis, episcleritis, erythema, always connected with phenomena of the inflammatory type and, in the case of Crohn's disease, also neurological complications.

There does not exist a definitive cure for such pathologies and the treatment is generally intended to reduce the negative effects of the acute phases of the disease and the maintenance of the remission phase. In particular, the treatment is generally of the pharmacological type in order to prevent any infections, for example, by means of antibiotics, or to reduce the inflammation, for example, by means of anti-inflammatory drugs and corticosteroids, the prolonged use of which involves, however, significant collateral effects and therefore they cannot be used for long-term treatments. An alternative pharmacology provides for the use of amino-salicylates, even if only a minority of patients is capable of continuing the treatment, for which many patients require immunosuppressive drugs.

In the most serious cases, it may further be necessary to resort to surgical interventions involving intestinal resection which may involve the colon and/or the rectum over some portions or also for the entire extent thereof. In the most general sector of medical treatments, it is known that the use of a number of compounds of n-butyric acid has advantageous biological effects on the digestive system, in particular the trophic function of the intestinal mucous membrane is known.

Butyric acid is a fatty monocarboxylic short-chained acid (with 4 carbon atoms), which is classified among the volatile fatty acids (AGV) together with acetic acid (chain with 2 carbon atoms) and propionic acid (chain with 3 carbon atoms), generally known as SCFA (Short-Chain Fatty Acids). Butyric acid has 2 isomers, n-butyric acid and isobutyric acid. At ambient temperature, n-butyric acid is in liquid form and further has a characteristic odour of rancid butter, perceived by human beings and by many species of animals even at very low concentrations.

The compounds of n-butyric acid of greater interest include the salts and the esters thereof, generally indicated as “butyrate”, and in particular the sodium salt thereof.

The ester of n-butyric acid is commercially available in liquid form while the sodium salt of the n-butyric acid is commercially available both in liquid form (in 50% aqueous solution—as a direct product of the synthesis reaction of the sodium salt from the n-butyric acid) and in solid granular form (in a white-coloured powder, stable up to 250° C.).

The compounds of n-butyric acid, depending on the surrounding environment, may be in a dissociated form or in an undissociated form; the latter form takes on a particular importance at a biological level because it can be absorbed by the intestinal walls and by the cell membranes of the microorganisms and may have a more pronounced effect with respect to the dissociated form.

The compounds of n-butyric acid are mainly produced from carbohydrates (cellulose and starch) by fermentation under anaerobic conditions by various microorganisms and this process also occurs in the large intestine. After its formation, the butyrate is partially metabolized while the non-metabolized fraction of the undissociated butyrate is absorbed in the large intestine and enters the circulatory system.

As set out above, the compounds of n-butyric acid have a trophic function of the intestinal mucous membrane with a resultant stimulation of the growth of intestinal villi.

The compounds of n-butyric acid further influence the development of some gastro-enteric microorganisms.

For example, data from literature report that the salts of volatile fatty acids may also inhibit the growth of 50% of the haemolytic strains of Escherichia coli (Galfi P., Neogrady S., 1992).

Other microorganisms which are sensitive to the salts of n-butyric acid are: Clostridium acetobutylicum, Escherichia coli, Streptococcus cremoris, Lactococcus lactis and cremoris, Salmonella spp. However, the compounds of n-butyric acid have some relevant disadvantages which limit the use and effectiveness thereof.

A first of these disadvantages is brought about by the extremely unpleasant odour of rancid butter which characterizes the initial n-butyric acid and which complicates the production and storage processes. Furthermore, the compounds of the n-butyric acid are particularly sensitive to acidic environments, where they can readily hydrolyze and reform the original n-butyric acid, for which reason, if they are administered per se, n-butyric acid would immediately form at a gastric level, making it no longer available for absorption thereof at an intestinal level.

In order to limit this disadvantage, it is known to micro-encapsulate the butyrate, coating it with a lipid matrix, as described, for example, in EP2352386, in the name of the same Applicant, which describes a production process for a compound based on n-butyric acid or the derivatives thereof which is/are micro-encapsulated in a lipid matrix which is based on saturated fatty acids and a mineral agent which allows the derivatives of the n-butyric acid to be protected from the highly acidic environment which is present at a gastric level, but allowing the release thereof at an enteric level, as a result of the degradation action of the lipid matrix by means of specific enzymes.

### DISCLOSURE OF THE INVENTION

The problem addressed by the present invention is to provide a novel composition for the treatment of Crohn's disease and/or ulcerative rectal colitis.

In the context of this problem, an object of the invention is to provide a composition for treating the pathologies which substantially act on the natural defences of the organism so as to limit or eliminate completely possible undesirable secondary effects.

This problem is solved and this object is achieved by the present invention by using a composition based on n-butyric acid or a derivative thereof, in micro-encapsulated form, for treating Crohn's disease and/or ulcerative colitis according to the appended claims.

In particular, the composition of the invention is in micro-encapsulated form and comprises a core based on n-butyric acid or based on a derivative thereof, and a coating which surrounds the core in which the coating is based on a lipid matrix which has a content of C14-C22 long chain saturated fatty acids between 40% and 95%.

As a result of this formulation, the n-butyric acid or the derivative thereof present in the core is released in a slow and controlled manner in the intestine and, as better demonstrated in the tests set out and discussed below, this release action of the n-butyric acid has brought a substantial improvement in the subjects affected by Crohn's disease and ulcerative rectal colitis.

Therefore, in a first aspect thereof, the invention relates to a composition in micro-encapsulated form, as specified above, for the treatment of ulcerative rectal colitis or Crohn's disease.

In a preferred manner, this composition is used for the treatment of ulcerative rectal colitis or Crohn's disease when they are in a light or moderate activity phase.

Furthermore, this composition is preferably used for the treatment of ulcerative rectal colitis or Crohn's disease with a dose of composition between 500 and 2000 mg/day, more preferably between 1000 and 1700 mg/day. In a preferred embodiment, the C14-C22 long chain saturated fatty acids which are present in the matrix are in the form of glycerides and constitute at least 80% by weight of the matrix.

The term “saturated” is not intended to be understood in an absolute sense, but instead it is intended to indicate fatty acids having a saturation level of at least 99%. Furthermore, it is particularly important for the fatty acids present in the matrix to be substantially present in the form of glycerides and not of free acids. To this end, it is necessary for the percentage of free acids within the lipid component of the matrix to be less than 10% and preferably for it to be less than 1%.

Preferably, the glycerides are in the form of triglycerides.

Preferably, the lipid component of the matrix according to the invention further has a content of C18 saturated fatty acid between 20% and 50% and a content of C16 saturated fatty acid between 50% and 75% with respect to the total of the saturated fatty acids which constitute the glycerides.

More preferably, the glycerides of saturated fatty acids are derived from hydrogenated palm oil.

The lipid component of the matrix is such as to have a melting temperature between 55° C. and 65° C. In a preferred embodiment, the matrix also comprises a mineral agent at a quantity between 1% and 20% with respect to the matrix.

In a preferred embodiment, there is present in the mineral agent a fraction (by weight) of calcium sulphate dihydrate, CaSO₄.2(H₂O), greater than 20%. In a greatly preferred manner, the fraction of calcium sulphate dihydrate in the mineral agent is greater than 50% and, in an even more preferable manner, the fraction of calcium sulphate dihydrate in the mineral agent is greater than 95%. The use of the calcium sulphate dihydrate is particularly important for obtaining a finished product which is provided with optimum characteristics of resistance to acidic environments.

In a preferred embodiment, the core of the micro-encapsulated composition is formed by granular material which is constituted by a salt or by an ester of the n-butyric acid and, in a greatly preferred manner, it is the sodium salt of n-butyric acid.

In a first embodiment, this granular material is based on powdered sodium butyrate, with a level of purity greater than 90-98%, having suitable particle size dimensions, for example, between 10 and 200 μm.

In a second embodiment, the granular material is composed of a powdered solid support, on which there is adsorbed an aqueous solution of sodium butyrate.

In a preferred manner, the solid support is of the inorganic type so as to withstand degrading phenomena for a longer time, and, in a greatly preferred manner, it is based on silica, with mean dimensions between 10 and 80 μm, preferably between 15 and 20 μm.

The quantity of silica used will be one which is sufficient to achieve the complete adsorption of the sodium butyrate, generally between 33% and 55% with respect to the aqueous solution of sodium butyrate.

The adsorption phase is carried out by mixing the aqueous solution, typically with a content of 50% of sodium butyrate, with the solid support in an admixture which is agitated at high speed and maintained at a temperature of approximately from 60 to 70° C.

The micro-encapsulated composition according to the present invention is preferably obtained by means of a spray cooling technique, in which the following steps are provided:
providing a granular material based on n-butyric acid or the derivative thereof,
mixing this granular material with the matrix based on lipid, bringing the admixture to a temperature greater than the melting temperature of the matrix,
spraying the admixture obtained in this manner in a cooling chamber having a temperature less than the melting temperature of the matrix so that the matrix solidifies around the granular material, coating it.

Preferably, the lipid component of the matrix is first caused to melt and then mixed with the granular material obtained by the adsorption of the liquid sodium butyrate on the silica or, alternatively, with the granular material formed by the solid sodium butyrate and with the mineral agent.

The mixing is preferably carried out in the presence of suitable emulsifying agents, such as, for example, esters of propylene glycol, so as to promote a homogeneous dispersion of the solid component (silica powder or solid sodium butyrate and mineral agent) in the molten lipid matrix.

Furthermore, there are preferably added to the admixture other polymer components based on cellulose and/or the derivatives thereof as inter alia stabilizing agents.

The mixing is carried out for approximately from 10 to 20 minutes until obtaining a homogeneous suspension of the solid granular material in the molten matrix.

In a variant of the invention, there is provision for one or more essential oils as aromatizing agents, antioxidants and anti-bactericides to be further added to the admixture in quantities from 0.1% to 5%.

Once the desired homogeneity has been obtained, the admixture is immediately injected at high pressure and by means of nozzles of a suitable shape inside a spray cooling chamber, in which the temperature is maintained between −2° C. and −12° C. so that, in the brief dwell time in air of the admixture particles, the lipid component of the matrix can advantageously solidify according to methods which are known per se (spray cooling technique). In this manner, there is obtained a solid granular product in micro-encapsulated form comprising an internal core which is formed by sodium butyrate (or by silica in which the aqueous solution thereof is adsorbed) and a covering and protecting coating of the internal core formed by the lipid matrix.

### EXAMPLE

The micro-encapsulated composition to which the invention relates has been subjected to a study in order to evaluate the therapeutic potential thereof in relation to Crohn's disease and ulcerative rectal colitis.

The study involved subjects who are affected by Crohn's disease and ulcerative rectal colitis and who were subdivided into two different groups (selected randomly), a first group treated with a micro-encapsulated composition of sodium butyrate and a second group treated with a placebo substance (corn starch).

The treatment lasted 12 weeks and provided for taking two capsules per day, each one containing 550 mg of a micro-encapsulated composition based on sodium butyrate (equal to approximately 200 mg of sodium butyrate per capsule) or an equal quantity of the placebo substance. The daily dose of micro-encapsulated composition based on sodium butyrate or placebo substance (excluding the weight of the capsule which contains it) was therefore approximately 1100 mg.

The micro-encapsulated composition based on sodium butyrate used in the study is commercially available under the trade name Butyrose , produced by the same Applicant.

All the subjects have been subjected just before the start of the treatment (time T0) and at the end of the treatment (time T1) to an examination of the faeces, directed towards analysing the microbial composition of the intestine (intestinal microbiota) and the presence of faecal calprotectin, a recognized biological indicator of intestinal inflammation.

Furthermore, all the subjects have expressed an evaluation of their quality of life before and after the treatment period, by means of an IBDQ questionnaire (Inflammatory Bowel Disease Questionnaire).

The results of the study are set out and commented upon below.

Intestinal microbiota analysis

The analysis was carried out by comparing the bactericidal strains prevalent at the time TO and at the time T1, after their identification by means of the method of Sparse Partial Least Squares Discriminant Analysis (SPLS-DA). In Table 1 below, there are set out the bacterial species prevalent at the time T0 and at the time T1 for the subjects who are affected by Crohn's disease and who are treated with the micro-encapsulated composition based on sodium butyrate.

TABLE 1

| Bacterial species | Per- | Bacterial species | Per- |
|---|---|---|---|
| prevalent at the time = T0 | centage | prevalent at the time = T1 | centage |
| Lachnospiraceae- | 2.55 | Ruminococcaceae- | 0.77 |
| Lachnoclostridium | | Subdoligranulum | |
| Ruminococcaceae- | 2.17 | Lachnospiraceae- | 0.57 |
| Flavonifractor plautii | | Blautia | |
| Desulfovibrionaceae- | 0.3 | Ruminocaccaceae- | |
| Bilophila_wadsworthia | | Butyricicoccus | 0.47 |
| Bacteroidaceae-Bacteroides | 2.41 | | |
| uniformis | | | |
| Fusobacteriaceae- | 2.59 | | |
| Fusobacterium-varium | | | |
| Streptococcaceae- | 2.79 | | |
| Streptococcus | | | |
| Erysipelotrichaceae- | 0.71 | | |
| Erysipelotrichaceae-UGC- | | | |
| 003 | | | |

An examination of Table 1 shows that, at the time T0 (just before the start of treatment), the prevalent bacterial species which are producers of SCFA (Lachnospiraceae—Lachnoclostridium and Ruminococcaceae—Flavonifractor p/autii) are equal to approximately 35% of the total of prevalent bacterial species while, at the time T1(end of treatment), the prevalent bacterial species are all producers of SCFA (100% of the total of prevalent bacterial species).

It can be seen particularly clearly that the prevalent bacterial species are highly butyrogenic.

In Table 2 below there are set out the bacterial strains prevalent at the time T0 and at the time T1for the subjects who are affected by ulcerative rectal colitis with the micro-encapsulated composition based on sodium butyrate.

TABLE 2

| Bacterial species | Per- | Bacterial species prev- | Per- |
|---|---|---|---|
| prevalent at the time = T0 | centage | alent at the time = T1 | centage |
| Bacteroidaceae- | 2.73 | Lachnospiraceae- | 0.81 |
| Bacteroides uniformis | | Lachnospira | |
| Streptococcaceae- | 0.3 | Lachnospiraceae- | 0.265 |
| Streptococcus | | Lachnospiraceae | |
| Ruminococcaceae- | 0.41 | NK4A136 group | |
| Ruminoclostridium-5 | | Lachnospiraceae- | 0.47 |
| Ruminococcaceae | 0.11 | Lachnoclostridium | |
| Erysipelotrichaceae- | 0.55 | Lachnospiraceae- | 0.44 |
| Erysipelotrichaceae UGC003 | | Lachnospiraceae UCG- | |
| Lachnospiraceae- | 0.57 | 010 | |
| Coporococcus | | | |
| Erysipelotrichaceae- | 0.04 | | |
| Turicibacter sanguinis | | | |

An examination of Table 2 shows that, at the time TO (just before the start of treatment), the prevalent bacterial species which are producers of SCFA (Ruminococcaceae-Ruminoclostridium-5, Ruminococcaceae and Lachnospiraceae-Coporococcus) are equal to approximately 23% of the total of prevalent bacterial species while, at the time T1(end of treatment), the prevalent bacterial species are all producers of SCFA (100% of the total of prevalent bacterial species).

In both groups of subjects treated with the micro-encapsulated composition based on sodium butyrate (both the ones affected by Crohn's disease and those affected by ulcerative rectal colitis), it has further been found how the variation of the intestinal microbiota has been particularly effective in subjects in which the activity of the disease was at a light or moderate level, while, in subjects in which the disease was in a remission phase, this effect was only slightly relevant.

Faecal calprotectin analysis

The analysis of the faecal calprotectin in subjects who are affected by Crohn's disease and who are subjected to treatment with the micro-encapsulated composition based on sodium butyrate has shown that, for approximately 67% of them, there has been a significant reduction (greater than or equal to 30% in patients with initial values greater than 250 μg/g) of the value of the faecal calprotection.

In subjects who are affected by Crohn's disease and who are subjected to treatment with the placebo, this significant reduction of the value of faecal calprotectin was found to be only 33% of the total.

The analysis of the faecal calprotectin in subjects who are affected by ulcerative rectal colitis and who are subjected to treatment with the micro-encapsulated composition based on sodium butyrate has not shown particular differences from the subjects subjected to treatment with a placebo.

Quality of life

All the subjects have expressed evaluations on their quality of life by means of responses to the IBDQ questionnaire (Inflammatory Bowel Disease Questionnaire) before and after the treatment.

The results collected and processed have shown a substantial increase which is statistically significant (p value=0.0071) of the quality of life in the subjects who are affected by ulcerative rectal colitis and who are treated with the micro-encapsulated composition based on sodium butyrate with respect to the subjects who are affected by the same pathology and who are treated with a placebo.

In the subjects who are affected by Crohn's disease, however, there have been found no appreciable differences in the evaluation of the quality of life between the subjects who are treated with the micro-encapsulated composition based on sodium butyrate and those treated with a placebo. In conclusion, the study has shown that a treatment with the micro-encapsulated composition based on sodium butyrate according to the present invention is effective in treating Crohn's disease and ulcerative rectal colitis, modifying the intestinal microbiota so as to promote and increase the species which produce SCFA and especially, in the case of Crohn's disease, of the bacterial species which produce n-butyric acid.

Furthermore, there has been found a substantial reduction in the intestinal inflammatory activity in the case of treatment of Crohn's disease and a significant increase in the subjective perception of the quality of life in the case of treatment of ulcerative rectal colitis.

1. A composition in micro-encapsulated form, comprising a core based on n-butyric acid or a derivative thereof, and a coating based on a lipid matrix which surrounds the core, in which the matrix has a content of C14-C22 long chain saturated fatty acids between 40% and 95%, for the treatment of ulcerative rectal colitis or Crohn's disease.

2. The composition according to claim 1, wherein the C14-C22 long chain saturated fatty acids which are present in the matrix are in the form of glycerides and constitute at least 80% by weight of the matrix.

3. The composition according to claim 2, wherein the glycerides of saturated fatty acids are derived from hydrogenated palm oil.

4. The composition according to claim 1, wherein the matrix comprises a mineral agent in an amount between 1% and 20% by weight, with respect to the matrix, the mineral agent comprising a fraction of calcium sulphate dihydrate greater than 20% by weight.

5. The composition according to claim 4, wherein the fraction of the calcium sulphate dihydrate in the mineral agent is greater than 50%.

6. The composition according to any onc of thc prcceding claims, claim 1 wherein the compound is the sodium salt of n-butyric acid.

7. The composition according to any onc of the preceding claims, claim 1 for the treatment of ulcerative rectal colitis or Crohn's disease in a light or moderate activity phase.

8. The composition according to any onc of thc prcceding claims, claim 7, wherein the treatment of ulcerative rectal colitis or Crohn's disease provides for a daily dose of the composition between 500 and 2000 mg/day.

9. The composition according to claim 8, wherein the treatment of ulcerative rectal colitis or Crohn's disease provides for a daily dose of the composition between 1000 and 1700 mg/day.
